(19) United States
(12) Patent Application Publication
(10) Pub. No.: US 2009/0269416 A1
(43) Pub. Date: Oct. 29, 2009

(54) Compositions and Methods For Preventing or Treating Chronic Renal Failure in Felines With Hyperthyroidism

(75) Inventors: Karen Joy Wedekind, St. Peters, MO (US); Timothy Arthur Allen, Lawrence, KS (US); Dale Allen Fritsch, Topeka, KS (US); Chadwick Everett Dodd, Lawrence, KS (US)

Correspondence Address: COLGATE-PALMOLIVE COMPANY, 909 RIVER ROAD, PISCATAWAY, NJ, 08855, US

(73) Assignee: HILL'S PET NUTRITION , INC., Topeka, KS (US)

(21) Appl. No.: 12/300,539

(22) PCT Filed: May 14, 2007
(86) PCT No.: PCT/US2007/011512

§ 371 Date: Jun. 23, 2009

Related U.S. Application Data
(60) Provisional application No. 60/800,066, filed on May 12, 2006.

(51) Int. Cl. A61K 33/42; A61K 33/24; A61K 33/18

(52) U.S. Cl. 424/601; 424/617; 424/667

(57) ABSTRACT

The invention relates to compositions for preventing chronic renal failure (CRF) in felines with hyperthyroidism wherein the compositions comprise (1) from about 28 to about 35% protein, the protein comprising at least about 75% vegetable protein, and (2) from about 0.1 to less than about 1 mg/kg iodine and/or from about 0.1 to about 1 mg/kg selenium. The invention also relates to compositions for treating CRF in hyperthyroid felines wherein the compositions comprise (1) from about 28 to about 35% protein, the protein comprising at least about 75% vegetable protein, and (2) from about 0.1 to less than about 0.3 mg/kg iodine and/or from about 0.1 to about 1 mg/kg selenium. The invention further relates to methods for preparing and using such compositions, articles of manufacture comprising such compositions, and means for communicating information about such compositions, methods, and articles of manufacture.

### CROSS REFERENCE TO RELATED APPLICATIONS

This present application claims priority to U.S. Provisional Application Ser. No. 60/800,066, tiled May 12, 2006 and PCT/US2007/011512, filed May 14, 2007 in the United States Patent and Trademark Office, the entireties of which are incorporated by reference.

### BACKGROUND OF THE INVENTION

1. Field of the Invention

The invention relates generally to compositions and methods for preventing or treating chronic renal failure (CRF) and particularly to compositions and methods for preventing or treating CRF in felines with hyperthyroidism.

2. Description of the Prior Art

Both hyperthyroidism and CRF (also known as chronic renal disease or renal insufficiency) are common diseases, particularly in older cats. Hyperthyroidism is characterized by hypermetabolism of the thyroid gland and excessive production of the thyroid hormones triiodothyronine (T₃ and tetraiodothyronine (T₄). Hyperthyroidism can mask concurrent renal disease by, for example, increasing glomerular flow rate (CRF). In addition, some anti-thyroid medications can cause kidney damage thereby resulting in the development of a kidney disease. CRF is a progressive, terminal kidney disease that is one of the leading causes of death in felines.

Thus, there is a need for compositions and methods for treating CRF in felines with hyperthyroidism that provide partial or complete relief. In addition, there is a need for compositions and methods for preventing CRF in felines with hyperthyroidism.

### SUMMARY OF THE INVENTION

It is an object of the invention to provide compositions suitable for preventing CRF in felines with hyperthyroidism.

It is another object of the invention to provide compositions suitable for treating CRF in felines with hyperthyroidism.

It is another object of the invention to provide methods for preventing CRF in felines with hyperthyroidism.

It is another object of the invention to provide methods for treating CRF in felines with hyperthyroidism.

It is another object of the invention to provide articles of manufacture comprising a composition of the invention or two or more ingredients that, when combined together and, optionally, with additional ingredients, yield a composition of the invention for preventing CRF in a feline with hyperthyroidism.

It is another object with hyperthyroidism to provide means for communicating information about the compositions, methods, and articles of manufacture of the invention.

Additional objects, features, and advantages of the invention will be apparent to those skilled in the art.

### DETAILED DESCRIPTION OF THE INVENTION

CRF is a progressive kidney disease that has four phases: loss of renal reserve, renal insufficiency, azotemia, and uremia. The kidneys have a large built-in reserve as only ˜30% of kidney capacity is needed for normal kidney function. Kidney capacity diminishes with time for a variety of reasons, for example, advanced age and diseases and medications that damage the kidney(s). Renal insufficiency is characterized by decrease in renal function, and is generally observed when about 70% of kidney function has been lost (i.e., when only about 30% of kidney capacity is available). Clinical signs are typically not obvious during the phases of loss of renal reserve and renal insufficiency, thereby making it difficult to detect CRF. Azotemia, the third phase of CRF, is characterized by an abnormally high concentration of urea, creatinine, and/or other non-protein nitrogen-containing substances in the blood. Renal function is markedly reduced in the azotemic phase, although clinical signs may be mild and can go undetected. Uremia is characterized by the presence of abnormal quantities of urine or urine constituents in blood. The uremic phase is characterized by overt clinical signs.

Hyperthyroidism is characterized by hypermetabolism of the thyroid gland and the excessive production of the thyroid hormones T₃ and T₄. Most of T₃ and T₄ are bound to serum proteins. The portion of T₃ and T₄ partitioned into serum, and not associated with protein, is called free T₃ (fT₃) and T₄ (fT₄). One skilled in the art can accurately diagnose hyperthyroidism in a feline utilizing thyroid function tests, examining clinical signs, and/or observing the animal's response to trial thyroid hormone administration. Thyroid function tests are known to those skilled in the art and include, for example, tests for determining the concentrations of total and free serum T₃ and T₄, tests for determining the concentration of thyroid stimulating hormone, and the sodium pertechnetate and T₃ suppression tests. See, for example, Small Animal Nutrition, pages 863-868 (2000).

CRF is a terminal disease in felines. It is, therefore, important to prevent decrease in renal function in felines that have been diagnosed with hyperthyroidism, but do not show clinical signs of CRF. Thus, in one aspect, the present invention provides a composition for preventing CRF in a feline with hyperthyroidism that may be susceptible to develop renal insufficiency. Such a feline is one that has not been diagnosed with CRF, but is at risk to develop CRF due to, for example, advanced age, genetic predisposition, or disease(s) or medication(s) that can damage the feline's kidney(s). Preventing CRF includes reducing the risk of, delaying the onset of, and/or keeping a hyperthyroid feline from developing CRF. Thus, feeding a composition for preventing CRF can reduce a feline's risk of developing CRF. In addition, feeding such a composition can slow down the progression of loss of a feline's kidneys' built-in reserve, thereby delaying the onset of the later phases of CRF.

The composition for preventing CRF is suitable for any feline with hyperthyroidism that is susceptible to develop CRF. In some embodiments, the feline is a companion feline. A companion feline can be a feline kept as a pet. A companion feline can also be a feline from a widely domesticated species, for example, cats (Felis domesticus) regardless of whether or not it is kept as a pet. In some embodiments, the feline is an adult feline. An adult feline is a feline of any age after juvenile growth and development has been completed, including senior and geriatric felines. For example, an adult cat typically is one that is from about one year old through the remainder of its life. A senior feline is one of an age at which it is at a risk for suffering from an age-related disease regardless of whether or not the feline shows obvious physical or behavioral signs of aging. For example, a senior cat typically is a cat from about seven to about eleven years old. A geriatric feline is a feline showing signs of aging. For example, a geriatric cat typically is a cat of about twelve years of age and beyond.

This composition for preventing CRF in a feline with hyperthyroidism comprises from about 28 to about 35% protein. The protein present in the composition comprises at least about 75% vegetable protein. The composition also comprises from about 0.1 to about 1 mg/kg iodine.

Commercial adult cat foods, for example, typically comprise from about 35 to about 45% protein. Thus, in many embodiments, a composition for preventing CRF in a hyperthyroid feline comprises less protein than most commercially available adult cat foods.

In some embodiments, the composition for preventing CRF in a hyperthyroid feline comprises from about 28 to about 31% protein. In some such embodiments, the composition comprises from about 28 to about 30% protein; and in other such embodiments, the composition comprises from about 29 to about 31% protein. In yet other such embodiments, the composition comprises from about 28 or about 28.5 to about 29, about 29.5, about 30, about 30.5, or about 31% protein.

In other embodiments, the composition for preventing CRF in a hyperthyroid feline comprises from about 31 to about 35% protein. In some such embodiments, the composition comprises from about 32 to about 34% protein. In other embodiments, the composition comprises from about 32 to about 35% protein. In other embodiments, the composition comprises from about 33 to about 35% protein. In other embodiments, die composition comprises from about 31, about 32, or about 33 to about 34 or about 35% protein. In further embodiments, the composition comprises from about 31 to about 32, about 33, about 34, or about 35% protein.

Dietary protein restriction is beneficial for preventing and treating CRF. Excessive protein is catabolized to urea and other nitrogenous compounds normally excreted by the kidneys. Decreased renal function leads to accumulation of these compounds. Endogenous proteins will be degraded if amino acid intake is insufficient to maintain nitrogen balance. Thus, a composition for preventing and/or treating CRF in a feline with hyperthyroidism can facilitate achieving nitrogen balance, and can help limit the accumulation of waste products by decreasing protein intake.

Because felines are carnivores, feline foods typically are formulated to comprise mostly animal and fish protein. To the contrary, the protein in a composition of the present invention for preventing CRF in a feline with hyperthyroidism comprises at least about 75% vegetable protein. In some such embodiments, the composition comprises at least about 80% vegetable protein. In other embodiments, the composition comprises at least about 85% vegetable protein. In other such embodiments, the protein comprises at least about 90% vegetable protein. And in other such embodiments, the protein comprises at least about 95% vegetable protein (i.e., from at least about 95 up to about 100% vegetable protein).

Thus, in some embodiments, the composition for preventing CRF in a feline with hyperthyroidism comprises from about 28 to about 31% protein wherein the protein comprises at least about 75% vegetable protein. In some such embodiments, the composition comprises from about 28 to about 30% protein wherein the protein comprises at least about 75, at least about 80, at least about 85, at least about 90, or at least about 95% vegetable protein. In other such embodiments, the composition comprises from about 29 to about 31% protein wherein the protein comprises at least about 75, at least about 80, at least about 85, at least about 90, or at least about 95% vegetable protein. In yet other such embodiments, the composition comprises from about 28 or about 28.5 to about 29, about 29.5, about 30, about 30.5, or about 31% protein wherein the protein comprises at least about 75, at least about 80, at least about 85, at least about 90, or at least about 95% vegetable protein.

In other embodiments, the composition for preventing CRF in a feline with hyperthyroidism comprises from about 31 to about 35% protein wherein the protein comprises at least about 75% vegetable protein. In some such embodiments, the composition comprises from about 31, about 32, or about 33 to about 34 or about 35% protein wherein the protein comprises at least about 75, at least about 80, at least about 85, at least about 90, or at least about 95% vegetable protein. In other embodiments, the composition comprises from about 31 to about 32, about 33, about 34, or about 35% protein, and the protein comprises at least about 75, at least about 80, at least about 85, at least about 90, or at least about 95% vegetable protein. In yet other embodiments, the composition comprises from about 28 or about 28.5 to about 29, about 29.5, or about 30% protein, and the protein comprises at least about 75, at least about 80, at least about 85, at least about 90, or at least about 95% vegetable protein.

In some embodiments, the composition for preventing CRF in a feline with hyperthyroidism comprises from about 0.1 to less than about 1 mg/kg iodine. In some such embodiments, the composition comprises from about 0.1 to about 0.5 mg/kg iodine. In other such embodiments, the composition comprises from about 0.1 to about 0.3 mg/kg iodine. In yet other such embodiments, the composition comprises from about 0.15 to about 0.25 mg/kg iodine. And in further such embodiments, the composition comprises from about 0.1 to about 0.2 mg/kg iodine. Iodine is a constituent of T₃ and T₄. The thyroid glands actively trap iodine to ensure an adequate supply of thyroid hormones. Iodine as used herein refers to the iodine atom without reference to its molecular or ionic form, and includes iodine present in one or more chemical forms such as, for example, iodide, iodate, and periodate.

In some embodiments, the composition of the invention for preventing CRF in a feline with hyperthyroidism comprises from about 0.1 to about 1 mg/kg selenium. In some such embodiments, the composition comprises from about 0.1 to about 0.8 mg/kg selenium. In other such embodiments, the composition comprises from about 0.15 to about 0.65 mg/kg selenium. In yet other such embodiments, the composition comprises from about 0.4 to about 0.7 mg/kg selenium. In further such embodiments, the composition comprises from about 0.3 to about 0.65 mg/kg selenium. Selenium has a role in maintaining normal thyroid and iodine metabolism, particularly through the control of the deiodinase enzymes that regulate the conversion of T₄ to T₃. Selenium as used herein refers to the selenium atom without reference to its molecular or ionic form, and includes selenium present in one or more chemical forms such as, for example, selenide, selenite, and selenate.

In some embodiments, the composition of the invention for preventing CRF in a feline with hyperthyroidism comprises from about 0.1 to less than about 1 mg/kg iodine as well as from about 0.1 to less than about 1 mg/kg selenium. In some such embodiments, the composition comprises from about 0.1 to about 0.5 mg/kg iodine and from about 0.1 to about 0.8 mg/kg selenium. In other such embodiments, the composition comprises from about 0.1 to about 0.3 mg/kg iodine and from about 0.15 to about 0.65 mg/kg selenium. In further such embodiments, the composition comprises (1) from about 0.1, about 0.15, or about 0.2 to about 0.25, about 0.3, or about 0.5 mg/kg iodine, and (2) from about 0.1, about 0.15, about 0.3, or about 0.4 to about 0.65, about 0.7, or about 0.8 mg/kg selenium.

Tables 2 and 3 of U.S. Patent Application No. US 2005/0058691 A1 list the iodine and selenium content of commercially available canned and dry cat foods. The average amounts of selenium in the 28 tested canned foods and 14 tested dry foods were 1.77 and 0.69 mg/kg. respectively, with many foods having more than 2 mg/kg. The average amounts of iodine in those foods were 7.83 mg/kg and 2.77 mg/kg, respectively, with some foods having more than 30 mg/kg. Thus, the compositions comprise amounts of iodine and/or selenium that are lower (and in some embodiments, much lower) than the amounts of iodine and selenium in many commercially available foods. Thus, in some embodiments, feeding a composition to a feline results in restricting the feline's intake of iodine. In other embodiments, feeding a composition to a feline results in restricting the feline's intake of selenium. In further embodiments, feeding a composition to a feline results in restricting the feline's intake of both iodine and selenium.

One skilled in the art would understand that a composition for preventing CRF in a feline with hyperthyroidism can comprise any of the combinations of protein (with varying amounts of vegetable protein), iodine, and selenium discussed above.

For example, in some embodiments, die composition for preventing CRF in a feline with hyperthyroidism comprises from about 29 to about 31% protein wherein the protein comprises at least about 75, at least about 80, at least about 85, at least about 90, or at least about 95% vegetable protein, and (1) from about 0.1, about 0.15, or about 0.2 to about 0.25, about 0.3, or about 0.5 mg/kg iodine, and/or (2) from about 0.1, about 0.15, about 0.3, or about 0.4 to about 0.65, about 0.7, or about 0.8 mg/kg selenium.

In other embodiments, die composition for preventing CRF in a feline with hyperthyroidism from about 28 or about 28.5 to about 29, about 29.5, about 30, about 30.5, or about 31% protein wherein the protein comprises at least about 75, at least about 80, at least about 85, at least about 90, or at least about 95% vegetable protein, and (1) from about 0.1, about 0.15, or about 0.2 to about 0.25, about 0.3, or about 0.5 mg/kg iodine, and/or (2) from about 0.1, about 0.15, about 0.3, or about 0.4 lo about 0.65, about 0.7, or about 0.8 mg/kg selenium.

In other embodiments, the composition for preventing CRF in a feline with hyperthyroidism comprises from about 31, about 32, or about 33 to about 34 or about 35% protein, wherein the protein comprises at least about 75, at least about 80, at least about 85, at least about 90, or at least about 95% vegetable protein, and (1) from about 0.1, about 0.15, or about 0.2 to about 0.25, about 0.3, or about 0.5 mg/kg iodine, and/or (2) from about 0.1, about 0.15, about 0.3, or about 0.4 to about 0.65, about 0.7, or about 0.8 mg/kg selenium.

In other embodiments, the composition for preventing CRF in a feline with hyperthyroidism comprises from about 28 or about 28.5 to about 29, about 29.5, or about 30% protein, and the protein comprises at least about 75, at least about 80, at least about 85, at least about 90, or at least about 95% vegetable protein, and (1) from about 0.1, about 0.15, or about 0.2 to about 0.25, about 0.3, or about 0.5 mg/kg iodine, and/or (2) from about 0.1, about 0.15, about 0.3, or about 0.4 to about 0.65, about 0.7, or about 0.8 mg/kg selenium.

Vegetarian diets generally contain higher amounts of fiber, magnesium, folic acid, vitamin C, vitamin D, and carotenoids than non-vegetarian diets. Vegetarian diets generally contain less saturated fat, cholesterol, protein, sulfur-containing amino acids, calcium, and iodine than non-vegetarian diets. Phosphorus from vegetable protein is less available than phosphorus from animal protein. Thus, some characteristics of vegetarian diets (e.g., lower amount of protein, lower bioavailability of phosphorus, lower amount of sulfur-containing amino acids thereby reducing diet acidity) are consistent with the goals of the invention to prevent and/or treat CRF in felines. Plant ingredients suitable for preparing compositions of die invention for preventing and/or treating CRF include, for example, potato concentrate, soy concentrate, soy protein isolate, soybean meal, com gluten meal, rice protein isolate, pea protein concentrate, wheat protein concentrate, and wheat protein isolate.

In some embodiments, the composition for preventing CRF in a feline with hyperthyroidism further comprises from about 0.45 to about 0.8% phosphorus. In some such embodiments, the composition comprises about 0.45 to about 0.6% phosphorus, for example, from about 0.45 to about 0.55% or from about 0.45 to about 0.5% phosphorus. In other such embodiments, the composition comprises from about 0.6 to about 0.8% phosphorus, for example, from about 0.7 to about 0.8% or from about 0.6 to about 0.75% phosphorus. Phosphorus as used herein refers to the phosphorus atom without reference to its molecular or ionic form. Secondary hyperparathyroidism and phosphorus retention have been incriminated as causes of CRF. Therefore, reduction of phosphorus in a feline's diet can be beneficial for preventing and/or treating CRF.

In some embodiments, the composition for preventing CRF in a feline with hyperthyroidism further comprises from about 0.2 to about 0.35% sodium. In some such embodiments, the composition comprises from about 0.2 to about 0.25% sodium. In other such embodiments, the composition comprises from about 0.25 to about 0.3% sodium. Sodium as used herein refers to die sodium atom without reference to its molecular or ionic form. When dietary sodium intake changes, fractional excretion of sodium must change as well to maintain sodium balance. Felines with CRF can only vary sodium excretion over a limited range, which narrows progressively as glomerular filtration rate declines. Thus, such felines cannot tolerate excessively high or excessively low dietary sodium intake. Feeding a feline a composition for preventing and/or treating CRF can help maintain optimal sodium balance.

In some embodiments, the composition for preventing CRF in a feline with hyperthyroidism further comprises from about 0.75 to about 1% potassium. In some such embodiments, the composition comprises from about 0.8 to about 1% potassium. In other such embodiments, the composition comprises from about 0.75 to about 0.9% potassium. Potassium as used herein refers to the potassium atom without reference to its molecular or ionic form. Normally, the majority of ingested potassium is excreted in the urine, with die remainder excreted in feces. The proportion of potassium excreted in feces increases in felines with CRF. Such felines typically also have disorders in potassium homeostasis. Feeding such felines a composition for preventing and/or treating CRF can help maintain optimal potassium balance.

In some embodiments, the composition for preventing CRF further comprises two or more of phosphorus, sodium, and potassium in any combination of the amounts discussed above.

In some embodiments, a composition for preventing CRF in a hyperthyroid feline as described above further comprises an antithyroid agent (i.e., the composition comprises one or more antithyroid agents). An antithyroid agent is a compound, a derivative thereof (e.g., a salt, solvate, or hydrate of the compound), or a composition comprising such compounds and/or derivatives that is used to treat hyperthyroidism. Suitable antithyroid agents include, for example, thioureylenes (e.g., methimazole, propylthiouracil, and carbimazole), aniline derivatives (e.g., sulfonamides), polyhydric phenols (e.g., resorcinol), and lithium salts. In some embodiments, the antithyroid agent comprises a thioureylene. Thioureylenes are five- or six-member thiourea derivatives that block production of thyroid hormones. In some such embodiments, the antithyroid agent comprises the thioureylene methimazole. In other such embodiments, the antithyroid agent comprises the thioureylene propylthiouracil. In further such embodiments, the antithyroid agent comprises the thioureylene carbimazole.

In some embodiments, a composition of the invention comprises a therapeutically-effective amount of an antithyroid agent (i.e., the composition comprises one or more antithyroid agents, and the total amount of the antithyroid agents is a therapeutically-effective amount). A therapeutically-effective or effective amount is an amount that will achieve the goal of treating the targeted condition. Those skilled in the art either know or can determine by routine experimentation how much of an agent or combination of agents to administer to a feline to treat a specific condition (e.g., hyperthyroidism). For example, one skilled in the art can prepare a composition that, when fed to the feline in a maintenance-sufficient amount, typically will deliver a therapeutically-effective amount of the agent(s) present in the composition. In some cases, the amount of the agent may vary with the stage of treatment. For example, higher doses of an antithyroid agent may be used in the initial stage of treatment (i.e., the therapeutically-effective amount of the particular agent may vary with the stage of the disease). One skilled in the art can prepare compositions with varying amounts of an antithyroid agent, and then feed those compositions sequentially to deliver the desired amount of antithyroid agent(s) to the feline.

Treatment can help a hyperthyroid feline with CRF to live a healthier, longer, and more active life. Thus, in another aspect, the invention provides a composition for treating CRF in a hyperthyroid feline in need of such treatment (i.e., die composition is useful for treating both CRF and hyperthyroidism). Treating CRF includes ameliorating, suppressing, and/or delaying CRF. One skilled in the art can diagnose CRF (as well as distinguish it from other diseases, for example, a kidney tumor, diabetes, and hyperthyroidism) by utilizing blood and urine tests as well as, for example, radiography, ultrasound, renal biopsy, and/or palpation. In addition to other symptoms, felines with CRF typically have an elevated serum creatinine. A composition for treating CRF is suitable for any hyperthyroid feline with CRF. Examples of felines that can be treated with a composition are discussed above in the context of the compositions for preventing CRF.

The composition for treating CRF in a feline with hyperthyroidism comprises from about 28 to about 30% protein. The protein present in the composition comprises at least about 75% vegetable protein. The composition also comprises from about 0.1 to less than about 1 mg/kg iodine and/or from about 0.1 to about 1 mg/kg iodine.

In some embodiments, the composition for treating CRF in a feline with hyperthyroidism comprises from about 28 to about 29% protein, and in other embodiments, the composition comprises from about 29 to about 30% protein. In yet other embodiments, the composition comprises from about 28 or about 28.5 to about 29, about 29.5, or about 30% protein.

In some embodiments, the composition for treating CRF in a feline with hyperthyroidism comprises at least about 80% vegetable protein. In other embodiments, the composition comprises at least about 85% vegetable protein. In other embodiments, the protein comprises at least about 90% vegetable protein. In other embodiments, the protein comprises at least about 95% vegetable protein (i.e., from at least about 95 up to about 100% vegetable protein). In some embodiments, the composition comprises from about 28 or about 28.5 to about 29, about 29.5, or about 30% protein wherein the protein comprises at least about 75, at least about 80, at least about 85, at least about 90, or at least about 95% vegetable protein.

In some embodiments, die composition for treating CRF in a feline with hyperthyroidism comprises from about 0.1 to less than about 1 mg/kg iodine. In some such embodiments, the composition comprises from about 0.1 to about 0.5 mg/kg iodine. In other such embodiments, the composition comprises from about 0.1 to about 0.3 mg/kg iodine. In yet other such embodiments, the composition comprises from about 0.15 to about 0.25 mg/kg iodine. And in further such embodiments, the composition comprises from about 0.1 to about 0.2 mg/kg iodine.

In some embodiments, the composition of the invention for treating CRF in a feline with hyperthyroidism comprises from about 0.1 to about 1 mg/kg selenium. In some such embodiments, the composition comprises from about 0.1 to about 0.8 mg/kg selenium. In other such embodiments, the composition comprises from about 0.15 to about 0.65 mg/kg selenium. In yet other such embodiments, the composition comprises from about 0.4 to about 0.7 mg/kg selenium. In further such embodiments, the composition comprises from about 0.3 to about 0.65 mg/kg selenium.

In some embodiments, the composition of the invention for treating CRF in a feline with hyperthyroidism comprises from about 0.1 to less than about 1 mg/kg iodine as well as from about 0.1 to less than about 1 mg/kg selenium. In some such embodiments, the composition comprises from about 0.1 to about 0.5 mg/kg iodine and from about 0.1 to about 08 mg/kg selenium. In other such embodiments, the composition comprises from about 0.1 to about 0.3 mg/kg iodine and from about 0.15 to about 0.65 mg/kg selenium. In further such embodiments, the composition comprises (1) from about 0.1, about 0.15, or about 0.2 to about 0.25, about 0.3, or about 0.5 mg/kg iodine, and (2) from about 0.1, about 0.15, about 0.3, or about 0.4 to about 0.65, about 0.7, or about 0.8 mg/kg selenium.

One skilled in die art would understand that a composition for treating CRF in a feline with hyperthyroidism can comprise any of the combinations of protein (with varying amounts of vegetable protein), iodine, and selenium discussed above.

For example, in some embodiments, the composition for treating CRF in a feline with hyperthyroidism comprises from about 28 or about 28.5 to about 29, about 29.5, or about 30% protein wherein the protein comprises at least about 75, at least about 80, at least about 85, at least about 90, or at least about 95% vegetable protein, and (1) from about 0.1, about 0.15, or about 0.2 to about 0.25, about 0.3, or about 0.5 mg/kg iodine, and/or (2) from about 0.1, about 0.15, about 0.3, or about 0.4 to about 0.65, about 0.7, or about 0.8 mg/kg selenium.

In some embodiments, the composition for treating CRF in a hyperthyroid feline further comprises from about 0.45 to about 0.6% phosphorus. In some such embodiments, the composition comprises from about 0.45 to about 0.55% phosphorus; and in other such embodiments from about 0.45 to about 0.5% phosphorus.

In some embodiments, the composition for treating CRF in a hyperthyroid feline further comprises from about 0.2 to about 0.25% sodium.

In some embodiments, the composition for treating CRF in a hyperthyroid feline further comprises from about 0.75 to about 1% potassium. In some such embodiments, the composition comprises from about 0.7 to about 0.9% potassium; and in other such embodiments from about 0.75 to about 0.85% potassium.

In some embodiments, the composition for treating CRF in a feline with hyperthyroidism further comprises two or more of phosphorus, sodium, and potassium in any combination of the amounts discussed above.

In some embodiments, a composition for treating CRF in a hyperthyroid feline as described above further comprises an antithyroid and/or an anti-CRF agent (i.e., the composition comprises one or more antithyroid agents and/or one or more anti-CRF agents). An anti-CRF agent is a compound, a derivative thereof (e.g., a salt, solvate, or hydrate of the compound), or a composition comprising such compounds and/or derivatives that is used to treat CRF. Suitable anti-CRF agents include, for example, agents that reduce blood pressure or protein loss in urine, anabolic steroids, antibiotics, agents that treat anemia, and agents that control vomiting. Suitable antithyroid agents are discussed above in the context of the compositions for treating CRF in a feline with hyperthyroidism. In some embodiments, a composition of the invention comprises a therapeutically-effective amount of an anti-CRF agent (i.e., the composition comprises one or more anti-CRF agents, and the total amount of the anti-CRF agents is a therapeutically-effective amount). In some embodiments, a composition of the invention for treating CRF in a feline with hyperthyroidism comprises a therapeutically-effective amount of an antithyroid agent. In some embodiments, a composition of the invention comprises a therapeutically-effective amount of an antithyroid agent and a therapeutically-effective amount of an anti-CRF agent.

In some embodiments, the compositions for preventing and/or treating CRF comprise a food composition (i.e., the compositions comprise one or more food compositions). In some embodiments, the compositions meet the AAFCO's minimum nutrient level requirements for reproduction or maintenance. In some embodiments, die food compositions comprise a dry food. In some embodiments, the food compositions comprise a semi-moist food. In some embodiments, the food compositions comprise a moist food. In some embodiments, the food compositions comprise a treat, snack, supplement, or partially or fully edible toy. In some embodiments, the compositions comprise a mixture of one or more foods.

In another aspect, the invention provides methods for preparing compositions for preventing and/or treating CRF in a feline with hyperthyroidism. Such composition can be prepared, for example, by mixing various ingredients (including food compositions) that, when combined, yield a composition of the invention. Compositions can also be prepared by the methods discussed in, for example, Small Animal Nutrition, pages 127-146 (2000). As discussed above, plant ingredients suitable for preparing compositions include, for example, potato concentrate, soy concentrate, soybean meal, soy protein isolate, corn gluten meal, rice protein isolate, pea protein concentrate, wheat protein concentrate, and wheat protein isolate. To prepare a low selenium-comprising composition of the invention, one can use, for example, a selenium-free mineral mix and ingredients that contain small amounts of selenium such as, for example, potato concentrate, soy concentrate, and soy protein isolate. To prepare a low iodine-comprising composition of the invention, one can, for example, avoid food colorings rich in iodine and can use an iodine-free mineral mix, non-iodized salt, or ingredients that contain small amounts of iodine such as, for example, potato concentrate, soy concentrate, and soy protein isolate. Iodine and selenium-containing ingredients suitable for preparing a composition of the invention are listed in, for example, table 3 of U.S. Patent Application No. US 2005/0058691 A1. Plant ingredients suitable for preparing a composition include, for example, soybean meal, com gluten meal, rice protein isolate, pea protein concentrate, wheat protein concentrate, and wheat protein isolate. Eggs can be used for preparing a composition as well. Meat (including fish) ingredients suitable for preparing a composition include, for example, pork liver, beef spleen, beef tongue, pork lung lobes, beef lung, meat protein isolate, deboned turkey, chicken backs, mackerel, oceanfish, and poultry by-product meal. One skilled in the art would understand that in many embodiments of the invention, one would need to utilize mostly plant ingredients to achieve high vegetable protein content as well as low iodine and/or selenium content. As discussed above, commercially available cat foods typically contain higher amounts of iodine and selenium than the amounts of iodine and/or selenium in a composition of the invention. To minimize iodine and/or selenium carryover, before preparing a composition of the invention, it may be desirable to clean the equipment that will be used. For example, to minimize iodine or selenium carryover from an earlier retort or extrusion run, the equipment can be appropriately flushed before a low iodine- and/or low selenium-comprising composition will be made. In some cases, it may also be desirable to discard the initial portion of a batch to obtain a composition with a consistent concentration of iodine and/or selenium throughout the entire batch.

In another aspect, the present invention provides a method for preventing CRF in a hyperthyroid feline susceptible to develop CRF. In some embodiments, the method comprises feeding the feline a composition selected from the compositions for preventing CRF in a hyperthyroid feline. One skilled in the art would understand that either a single composition can be fed to the feline or, alternatively, different such compositions can be fed to the feline for varying time intervals.

In some embodiments, the method for preventing CRF in a feline with hyperthyroidism comprises feeding the feline a composition comprising (1) from 28 to about 35% protein wherein the protein comprises at least about 75% vegetable protein, and (2) from about 0.1 to less than about 1 mg/kg iodine and/or from about 0.1 to about 1 mg/kg selenium.

In some such embodiments, the method for preventing CRF in a hyperthyroid feline comprises feeding the feline a composition comprising from about 29 to about 31% protein wherein the protein comprises at least about 75, at least about 80, at least about 85, at least about 90, or at least about 95% vegetable protein, and (1) from about 0.1, about 0.15, or about 0.2 to about 0.25, about 0.3, or about 0.5 mg/kg iodine, and/or (2) from about 0.1, about 0.15, about 0.3, or about 0.4 to about 0.65, about 0.7, or about 0.8 mg/kg selenium.

In some such embodiments, the method for preventing CRF in a hyperthyroid feline comprises feeding the feline a composition comprising from about 28 or about 28.5 to about 29, about 29.5, about 30, about 30.5, or about 31% protein wherein the protein comprises at least about 75, at least about 80, at least about 85, at least about 90, or at least about 95% vegetable protein, and (1) from about 0.1, about 0.15, or about 0.2 to about 0.25, about 0.3, or about 0.5 mg/kg iodine, and/or (2) from about 0.1, about 0.15, about 0.3, or about 0.4 to about 0.65, about 0.7, or about 0.8 mg/kg selenium.

In other such embodiments, die method for preventing CRF in a feline with hyperthyroidism comprises feeding the feline a composition comprising from about 31, about 32, or about 33 to about 34 or about 35% protein wherein the protein comprises at least about 75, at least about 80, at least about 85, at least about 90, or at least about 95% vegetable protein, and (1) from about 0.1, about 0.15, or about 0.2 to about 0.25, about 0.3, or about 0.5 mg/kg iodine, and/or (2) from about 0.1, about 0.15, about 0.3, or about 0.4 to about 0.65, about 0.7, or about 0.8 mg/kg selenium.

In other such embodiments, the method for preventing CRF in a feline with hyperthyroidism comprises feeding the feline a composition comprising from about 31 to about 32, about 33, about 34, or about 35% protein, and the protein comprises at least about 75, at least about 80, at least about 85, at least about 90, or at least about 95% vegetable protein, and (1) from about 0.1, about 0.15, or about 0.2 to about 0.25, about 0.3, or about 0.5 mg/kg iodine, and/or (2) from about 0.1, about 0.15, about 0.3, or about 0.4 to about 0.65, about 0.7, or about 0.8 mg/kg selenium.

In other such embodiments, hyperthyroidism comprises from about 28 or about 28.5 to about 29, about 29.5, or about 30% protein, and the protein comprises at least about 75, at least about 80, at least about 85, at least about 90, or at least about 95% vegetable protein, and (1) from about 0.1, about 0.15, or about 0.2 to about 0.25, about 0.3, or about 0.5 mg/kg iodine, and/or (2) from about 0.1, about 0.15, about 0.3, or about 0.4 to about 0.65, about 0.7, or about 0.8 mg/kg selenium.

In some embodiments of the method for preventing CRF in a feline with hyperthyroidism, the composition that is fed to the feline further comprises from about 0.45 to about 0.8% phosphorus. In some such embodiments, the composition that is fed to the feline comprises from about 0.45 to about 0.6% phosphorus, for example, from about 0.45 to about 0.55% or from about 0.45 to about 0.5% phosphorus. In other such embodiments, the composition fed to the feline comprises from about 0.6 to about 0.8% phosphorus, for example, from about 0.7 to about 0.8% or from about 0.6 to about 0.75% phosphorus.

In some embodiments of the method for preventing CRF in a feline with hyperthyroidism, the composition that is fed to the feline further comprises from about 0.2 to about 0.35% sodium. In some such embodiments, the composition fed to the feline comprises from about 0.2 to about 0.25% sodium, and in other such embodiments, the composition fed to the feline comprises from about 0.2 to about 0.25% sodium.

In some embodiments of the method for preventing CRF in a feline with hyperthyroidism, the composition that is fed to the feline further comprises from about 0.75 to about 1% potassium. In some such embodiments, the composition fed to the feline comprises from about 0.8 to about 1% potassium, and in other such embodiments, the composition comprises from about 0.75 to about 0.9% potassium.

In some embodiments of the method for preventing CRF in a feline with hyperthyroidism, the composition fed to the feline further comprises two or more of phosphorus, sodium, and potassium in any combination of the amounts discussed above.

In another aspect, the present invention provides a method for treating CRF in a hyperthyroid feline in need of such treatment. In some embodiments, the method comprises feeding the feline a composition selected from the compositions for treating CRF in a hyperthyroid feline. One skilled in the art would understand that either a single composition can be led to the feline or, alternatively, different such compositions can be fed to the feline for varying time intervals.

In some embodiments, the method for preventing CRF in a feline with hyperthyroidism comprises feeding the feline a composition comprising (1) from 28 to about 35% protein wherein the protein comprises at least about 75% vegetable protein, and (2) from about 0.1 to less than about 1 mg/kg iodine and/or from about 0.1 to about 1 mg/kg selenium.

In some such embodiments, the method comprises feeding the feline a composition comprising from about 28 or about 28.5 to about 29, about 29.5, or about 30% protein wherein the protein comprises at least about 75, at least about 80, at least about 85, at least about 90, or at least about 95% vegetable protein, and (1) from about 0.1, about 0.15, or about 0.2 to about 0.25, about 0.3, or about 0.5 mg/kg iodine, and/or (2) from about 0.1, about 0.15, about 0.3, or about 0.4 to about 0.65, about 0.7, or about 0.8 mg/kg selenium.

In some embodiments of the method for treating CRF in a feline with hyperthyroidism, the composition that is fed to the feline further comprises from about 0.45 to about 0.6% phosphorus. In some such embodiments, the composition fed to the feline comprises from about 0.45 to about 0.55% phosphorus. In other such embodiments, the composition fed to the feline comprises from about 0.45 to about 0.5% phosphorus.

In some embodiments of the method for treating CRF in a feline with hyperthyroidism, the composition that is fed to the feline further comprises from about 0.2 to about 0.25% sodium.

In some embodiments of the method for treating CRF in a feline with hyperthyroidism, the composition that is fed to the feline further comprises from about 0.75 to about 1% potassium. In some such embodiments, the composition that is fed to the feline comprises from about 0.7 to about 0.9% potassium. In other such embodiments, the composition fed to the feline comprises from about 0.75 to about 0.85% potassium.

In some embodiments of the method for treating CRF in a feline, the composition fed to the feline further comprises two or more of phosphorus, sodium, and potassium in any combination of the amounts discussed above.

In some embodiments, die methods for prevention and treatment of CRF in a feline with hyperthyroidism further comprise administering to die feline an anti-CRF agent. In some embodiments, the methods for prevention and treatment of CRF in a feline with hyperthyroidism further comprise administering to the feline an antithyroid agent. In some embodiments, the methods for prevention and treatment of CRF in a feline with hyperthyroidism further comprise administering to the feline an anti-CRF agent and an antithyroid agent. Such agents are administered in conjunction with feeding a composition of the invention. The term “in conjunction” means that an agent is administered to the feline either together with a composition of the invention or separately from the composition at the same or different frequency via the same or different administration route and either at about the same time as the composition or periodically. “Administering” means that the agent is introduced in a suitable dosage form into the feline by a suitable administration route, for example, orally, topically, or parenterally. “About at the same time” generally means that an agent is administered when a composition is fed to the feline or within about 72 hours of feeding the composition to the feline. “Periodically” generally means that an agent is administered to a feline following a dosage schedule suitable for administering the agent while a composition is fed to the feline routinely as appropriate for that feline. Thus, the term “in conjunction” specifically includes situations when an agent is administered to a feline for a prescribed period of time while a composition is fed to the feline for a much longer period of time (e.g., for life). If more than one agent is administered, the dosage form and route of administration for each agent may vary. In addition, one composition may be substituted with another composition while a specific agent is administered to the feline.

Suitable anti-CRF and antithyroid agents are discussed above in the context of the compositions of the invention. Such agents can be administered, for example, in the form of salts derived from inorganic or organic acids. Depending on the particular compound, a salt of die compound may be advantageous due to one or more of the salt's physical properties, for example, enhanced pharmaceutical stability in differing temperatures and humidities, or a desirable solubility in water or oil. The salt preferably is a pharmaceutically-acceptable salt.

The total daily dose of an agent (administered in single or divided doses) is typically from about 0.001 to about 100 mg/kg, preferably from about 0.001 to about 30 mg/kg, and even more preferably from about 0.01 to about 10 mg/kg body weight. Dosage unit compositions can contain such amounts or submultiples thereof to make up the daily dose. In many instances, the administration of an agent will be repeated a plurality of times. Multiple doses per day typically may be used to increase the total daily dose. Factors affecting the preferred dosage regimen include, for example, the age, weight, and condition of the feline; the severity of the disease; the route of administration; pharmacological considerations (e.g., activity, efficacy, and pharmacokinetic and toxicology profiles of die particular agent); whether a drug delivery system is utilized; and whether the agent is administered as part of a drug combination. Thus, the dosage regimen actually employed can vary widely, and can differ from the dosage regimen set forth above.

In another aspect, the invention provides a use of a composition (i.e., any composition for preventing CRF in a feline discussed above) to prepare a medicament to prevent CRF in a feline with hyperthyroidism.

In another aspect, the invention provides a use of a composition (i.e., any composition for treating CRF in a feline as discussed above) to prepare a medicament to treat CRF in a feline.

In a further aspect, the invention provides an article of manufacture, for example, a kit comprising a composition of the invention as discussed above. The kit is suitable for preventing and/or treating CRF in a feline with hyperthyroidism. In some embodiments, the kit further comprises an anti-CRF agent (i.e., one or more anti-CRF agents). In some embodiments, the kit further comprises an antithyroid agent (i.e., one or more antithyroid agents). In some embodiments, the kit further comprises an anti-CRF agent and an antithyroid agent. In some embodiments, the kit further comprises instructions for, for example, one or more of (a) feeding the composition to a feline with hyperthyroidism, (b) administering an anti-CRF agent and/or an antithyroid agent to the feline in conjunction with feeding the feline the composition, (c) preventing CRF in a feline with hyperthyroidism by feeding the feline the composition, (d) treating CRF in a feline with hyperthyroidism by feeding the feline the composition, (e) preventing CRF in a feline with hyperthyroidism by administering to the feline an anti-CRF agent and/or an antithyroid agent in conjunction with feeding the feline the composition, and (f) treating CRF in a feline with hyperthyroidism by administering to the feline an anti-CRF agent and/or an antithyroid agent in conjunction with feeding die feline the composition.

In some embodiments of the invention, the kit comprises two or more ingredients that, when combined together and optionally with additional ingredients that are or are not a part of die kit, yield a composition of the present invention. If such additional ingredients are to be used, the kit provides instructions about these ingredients. In some embodiments, the kit further comprises an anti-CRF agent. In some embodiments, the kit further comprises an antithyroid agent. In some embodiments, the kit further comprises an anti-CRF agent and an antithyroid agent. In some embodiments, the kit further comprises instructions for one or more of (a) preparing a composition of the invention for preventing CRF in a hyperthyroid feline by combining the ingredients, (b) preparing a composition for treating CRF in a hyperthyroid feline by combining the ingredients, (c) feeding a feline with hyperthyroidism a composition for preventing CRF, (d) feeding a feline with hyperthyroidism a composition for treating CRF, (e) administering an anti-CRF agent and/or an antithyroid agent to a feline in conjunction with feeding the feline a composition for preventing CRF, (f) administering an anti-CRF agent and/or an antithyroid agent to a feline with hyperthyroidism in conjunction with feeding the feline a composition for treating CRF, (g) preventing CRF in a feline with hyperthyroidism by feeding the feline a composition for preventing CRF, (h) treating CRF in a feline with hyperthyroidism by feeding the feline a composition for treating CRF, (i) preventing CRF in a feline with hyperthyroidism by administering to the feline an anti-CRF agent and/or an antithyroid agent in conjunction with feeding the feline a composition for preventing CRF, and (j) treating CRF in a feline with hyperthyroidism by administering to die feline an anti-CRF agent and/or an antithyroid agent in conjunction with feeding the feline a composition for treating CRF.

In some embodiments, the kit comprises in separate containers in a single package or in separate containers in a virtual package, as appropriate, a composition or two or more ingredients, that, when combined together and optionally with additional ingredients that are or are not a part of the kit, yield a composition of the invention, and instructions for one or more of (a) preparing a composition of the invention for preventing CRF in a feline with hyperthyroidism by combining the ingredients, (b) preparing a composition for treating CRF in a feline with hyperthyroidism by combining the ingredients, (c) feeding a feline with hyperthyroidism a composition to prevent CRF, (d) feeding a feline with hyperthyroidism a composition to treat CRF, (e) administering an anti-CRF agent to a feline with hyperthyroidism in conjunction with feeding die feline a composition to prevent CRF, (f) administering an anti-CRF agent and/or an antithyroid agent to a feline with hyperthyroidism in conjunction with feeding the feline a composition to treat CRF, (g) preventing CRF in a feline with hyperthyroidism by feeding the feline a composition for preventing CRF, (h) treating CRF in a feline with hyperthyroidism by feeding the feline a composition for treating CRF, (i) preventing CRF in a feline with hyperthyroidism by administering to die feline an anti-CRF agent and/or an antithyroid agent in conjunction with feeding the feline a composition for preventing CRF, and (j) treating CRF in a feline with hyperthyroidism by administering to the feline an anti-CRF agent and/or an antithyroid agent in conjunction with feeding the feline a composition for Seating CRF.

The term “single package” generally means that the components of a kit are physically associated in or with one or more containers and considered as a unit of manufacture, distribution, sale, or use. Containers include, for example, bags, boxes, bottles, shrink wrap packages, stapled or otherwise fixed components, and combinations thereof. A single package can be, for example, containers or individual food compositions physically associated such that they are considered a unit for manufacture, distribution, sale, or use. The term “virtual package” generally means that die components of a kit are associated by directions on one or more physical or virtual kit components instructing the user how to obtain additional components, e.g., in a bag containing one component and directions instructing the user to go to a website, contact a recorded message, view a visual message, or contact a caregiver to obtain instructions on how to use the kit. When the kit comprises a virtual package, the kit is limited to instructions in a virtual environment with one or more physical kit components.

In some embodiments, the kit is suitable for preventing chronic renal failure in a feline with hyperthyroidism and comprises in separate containers in a single package or in separate containers in a virtual package, as appropriate, a composition comprising (1) from about 28 to about 35% protein wherein the protein comprises at least about 75% vegetable protein, and (2) from about 0.1 to less than about 1 mg/kg iodine and/or from about 0.1 to about 1 mg/kg selenium, or two or more ingredients that, when combined together and, optionally, with additional ingredients that are or are not a part of the kit, yield a composition comprising from about 28 to about 35% protein wherein the protein comprises at least about 75% vegetable protein, and (2) from about 0.1 to less than about 1 mg/kg iodine and/or from about 0.1 to about 1 mg/kg selenium, and one or more of (a) an anti-chronic renal failure agent, (b) antithyroid agent, (c) instructions for preparing the composition, (d) instructions for feeding the composition to a feline with hyperthyroidism, (e) instructions for preventing chronic renal failure in a feline with hyperthyroidism by feeding die feline die composition, and (f) instructions for preventing chronic renal failure in a feline with hyperthyroidism by administering to the feline an anti-chronic renal failure agent and/or an antithyroid agent in conjunction with feeding the feline the composition. In some such embodiments, the kit comprises a composition comprising (1) from about 31 to about 35% protein wherein the protein comprises at least about 75% vegetable protein, and (2) from about 0.1 to less than about 1 mg/kg iodine and/or from about 0.1 to about 1 mg/kg selenium, or two or more ingredients that, when combined together and, optionally, with additional ingredients that are or are not a part of the kit, yield a composition comprising (1) from about 31 to about 35% protein wherein the protein comprises at least about 75% vegetable protein, and (2) from about 0.1 to less than about 1 mg/kg iodine and/or from about 0.1 to about 1 mg/kg selenium.

In other embodiments, the kit is suitable for treating chronic renal failure in a feline with hyperthyroidism and comprises in separate containers in a single package or in separate containers in a virtual package, as appropriate, a composition comprising (1) from about 28 to about 30% protein wherein the protein comprises at least about 75% vegetable protein, and (2) from about 0.1 to less than about 1 mg/kg iodine and/or from about 0.1 to about 1 mg/kg selenium, or two or more ingredients that, when combined together and, optionally, with additional ingredients that are or are not a part of the kit, yield a composition comprising from (1) from about 28 to about 30% protein wherein the protein comprises at least about 75% vegetable protein, and (2) from about 0.1 to less than about 1 mg-kg iodine and/or from about 0.1 to about 1 mg/kg selenium, and one or more of (a) an anti-chronic renal failure agent, (b) an antithyroid agent, (c) instructions for preparing the composition, (d) instructions for feeding the composition to a feline with hyperthyroidism, (e) instructions for treating chronic renal failure in a feline with hyperthyroidism by feeding the feline the composition, and (f) instructions for treating chronic renal failure in a feline with hyperthyroidism by administering to the feline an anti-chronic renal failure agent and/or an antithyroid agent in conjunction with feeding the feline the composition.

In a further aspect, die invention provides a means for communicating information about or instructions for one or more of (a) feeding a feline with hyperthyroidism composition to prevent CRF, (b) feeding a feline with hyperthyroidism a composition to treat CRF, (c) administering an anti-CRF agent and/or an antithyroid agent to a feline in conjunction with feeding the feline a composition to prevent CRF, (d) administering an anti-CRF agent and/or an antithyroid agent to a feline with hyperthyroidism in conjunction with feeding the feline a composition to treat CRF, (e) preventing CRF in a feline with hyperthyroidism by feeding the feline a composition for preventing CRF, (f) treating CRF in a feline with hyperthyroidism by feeding the feline a composition for treating CRF, (g) preventing CRF in a Celine with hyperthyroidism by administering to the feline an anti-CRF agent and/or antithyroid agent in conjunction with feeding the feline a composition for preventing CRF, (h) treating CRF in a feline with hyperthyroidism by administering to the feline an anti-CRF agent and/or antithyroid agent in conjunction with feeding the feline a composition for treating CRF, (i) using a kit to prevent CRF in a feline with hyperthyroidism, and (j) using a kit to treat CRF in a feline with hyperthyroidism. The communication means comprise, for example, a document, digital storage media, optical storage media, audio presentation, or visual display containing the information or instructions. Preferably, the communication means is a displayed web site or a brochure, product label, package insert, advertisement, or visual display containing such information or instructions. Useful information or instructions include, for example, (1) information and instructions how to use a composition, method, or kit of the invention and (2) contact information for animal caregivers if they have a question about the invention and its uses.

In some embodiments, the present invention provides a means for communicating information about or instructions for one or more of (a) preventing chronic renal failure in a feline with hyperthyroidism by feeding the feline a composition comprising (1) from about 28 to about 35% protein wherein the protein comprises at least about 75% vegetable protein, and (2) from about 0.1 to less than about 1 mg/kg iodine and/or from about 0.1 to about 1 mg/kg selenium; (b) preventing chronic renal failure in a feline with hyperthyroidism by administering to the feline an anti-chronic renal failure agent and/or an antithyroid agent in conjunction with feeding the feline a composition comprising (1) from about 28 to about 35% protein wherein the protein comprises at least about 75% vegetable protein, and (2) from about 0.1 to less than about 1 mg/kg iodine and/or from about 0.1 to about 1 mg/kg selenium; and (c) using a kit comprising a composition comprising (1) from about 28 to about 35% protein wherein the protein comprises at least about 75% vegetable protein, and (2) from about 0.1 to less than about 1 mg/kg iodine and/or from about 0.1 to about 1 mg/kg selenium, or two or more ingredients that when combined together and, optionally, with additional ingredients, yield a composition comprising (1) from about 28 to about 35% protein wherein the protein comprises at least about 75% vegetable protein, and (2) from about 0.1 to less than about 1 mg/kg iodine and/or from about 0.1 to about 1 mg/kg selenium. The means comprise a document, digital storage media, audio presentation, or visual display containing the information or instructions.

In other embodiments, the present invention provides a means for communicating information about or instructions for one or more of (a) treating chronic renal failure in a feline with hyperthyroidism by feeding the feline a composition comprising (1) from about 28 to about 30% protein wherein the protein comprises at least about 75% vegetable protein, and (2) from about 0.1 to less than about 1 mg/kg iodine and/or from about 0.1 to about 1 mg/kg selenium; (b) treating chronic renal failure in a feline with hyperthyroidism by administering to the feline and anti-chronic renal failure agent and/or an antithyroid agent in conjunction with feeding the feline a composition comprising (1) from about 28 to about 30% protein wherein the protein comprises at least about 75% vegetable protein, and (2) from about 0.1 to less than about 1 mg/kg iodine and/or from about 0.1 to about 1 mg/kg selenium; and (c) using a kit comprising a composition comprising (1) from about 28 to about 30% protein wherein the protein comprises at least about 75% vegetable protein, and (2) from about 0.1 to less than about 1 mg/kg iodine and/or from about 0.1 to about 1 mg/kg selenium, or two or more ingredients that when combined together and, optionally, with additional ingredients, yield a composition comprising (1) from about 28 to about 30% protein wherein the protein comprises at least about 75% vegetable protein, and (2) from about 0.1 to less than about 1 mg/kg iodine and/or from about 0.1 to about 1 mg/kg selenium. The communication means comprise a document, digital storage media, audio presentation, or visual display containing the information or instructions.

### EXAMPLES

The invention can be further illustrated by the following examples, although it will be understood that the examples are included merely for purposes of illustration and are not intended to limit the scope of the invention unless otherwise specifically indicated.

### Example 1

Examples 1 illustrates die effect of the compositions of the invention on preventing and/or treating CRF concurrently with treating hyperthyroid cats.

Food B1 was formulated as a dry cat food containing 0.6% selenium. Food B1 comprised soybean meal, corn gluten meal, poultry meal, and pork meat protein isolate as the protein ingredients, and was formulated with both iodized and non-iodized salt but no selenium. The average moisture content was 6.6%, the average protein content was 33.7%, and the average iodine content was 0.38 mg/kg (ten samples were taken for iodine analysis, and the iodine content of those samples varied from 0.27 to 0.60 mg/kg).

Food 2 was formulated as a wet cat food containing 0.6% selenium. It comprised soybean meal, pork lungs, chicken, and pork liver as the protein ingredients. No source of iodine was intentionally added to food B2. No selenium salts were added to food B1. The average moisture content for those samples was 78.2%, the average protein content was 33.2%, and the average iodine content was 0.21 mg/kg (ten samples were taken for iodine analysis, and the iodine content of those samples varied from 0.14 to 0.27 mg/kg).

Food B1 and food B2 were mixed in a 1:1 ratio, thus resulting in food B. A group of five cats diagnosed with hyperthyroidism was fed food B for six weeks. Another live cats diagnosed with hyperthyroidism were given 2.5 mg methimazole orally once a day for six weeks while they were fed food B. Eight eats diagnosed with hyperthyroidism were given 2.5 mg methimazole orally once a day for six weeks while they were fed a commercially available cat food. The thyroid hormone profiles and serum chemistries of all cats were measured at zero, two, four, and six weeks. All cats enrolled in the study had elevated total T₄ and/or free T₄, with the majority of the cats exhibiting one or more clinical signs associated with hyperthyroid disease (e.g., weight loss, heart murmur/tachycardia, unkempt hair coat, thyroid gland enlargement, increased appetite, vomiting, increased activity, diarrhea, polyuria/polydipsia, aggressiveness, and panting). The results from the study are presented in Tables 1 and 2.

TABLE 1
Change in Serum Total T₄ Levels

| | Change in Serum Total |
|---|---|
| Treatment | T₄ Levels (nmol/L) |
| Methimazole | −20.3 |
| Food B | −31.5 |
| Methimazole + Food B | −42.2 |

TABLE 2
Change in Serum Creatinine Levels

| | Change in Serum |
|---|---|
| Treatment | Creatinine Levels (mg/dL) |
| Methimazole | +0.46 |
| Food B | −0.08 |
| Methimazole + Food B | −0.03 |

Treatment with 2.5 mg methimazole administered orally once daily for six weeks resulted in a decrease in serum total T₄ concentration and in an increase in serum creatinine concentration (serum creatinine was a bit higher than die 0.8-1.8 mg/dL normal creatinine concentration range). Although this treatment was effective (the total T₄ concentration was lowered although it did not reach the normal total T₄ concentration range of 10-55 nmol/dL). it may cause or exacerbate kidney disease, and may therefore be unsuitable for treating hyperthyroidism in cats with CRF.

Feeding food B resulted in a greater decrease in serum total T₄ concentration than treatment with 2.5 mg methimazole alone (although die total T₄ concentration was still a bit higher than the normal total T₄ concentration range). It also resulted in a decrease in serum creatinine concentration (with creatinine concentration falling within the normal creatinine range). Feeding food B is a more effective treatment for hyperthyroidism than administering 2.5 mg methimazole alone. Feeding food B does not result in the side effects associated with methimazole treatment. Food B is also suitable for felines suffering from or susceptible to developing CRF.

Administering 2.5 mg methimazole while feeding food B resulted in the greatest decrease in serum total T₄ concentration. That decrease in serum total T₄ concentration was higher than the corresponding decrease for treatment with methimazole only with T₄ falling within the normal total T₄ concentration range, indicating that there is a synergism between the action of food B and the antithyroid agent. Administering methimazole in conjunction with feeding food B also resulted in a decrease in serum creatinine concentration (with serum creatinine concentration within the normal range), indicating that the combination is suitable for felines with or susceptible to developing CRF.

### Example 2

Examples 2 illustrates the effect of the compositions of the invention on preventing and/or treating CRF concurrently with treating hyperthyroidism in cats.

Fifteen cats diagnosed with hyperthyroidism were fed food B (from Example 1) for twelve weeks. Eleven cats diagnosed with hyperthyroidism were fed a commercially available cat food (i.e., control food) for twelve weeks. The thyroid hormone profiles and serum chemistries of all cats were measured at zero, two, four, six. and twelve weeks. All cats enrolled in the study had elevated total T₄ and/or free T₄, with the majority of the cats exhibiting one or more clinical signs associated with hyperthyroid disease. The results from this study are presented in Tables 3 and 4.

TABLE 3
Change in Serum Total T₄ Levels

| | Change in Serum Total |
|---|---|
| Treatment | T₄ Levels (nmol/L) |
| Control Food | +6.5 |
| Food B | −50.4 |

TABLE 4
Change in Serum Creatinine Levels

| | Change in Serum |
|---|---|
| Treatment | Creatinine Levels (mg/dL) |
| Control Food | +0.282 |
| Food B | −0.277 |

Feeding control food for twelve weeks resulted in an increase in both serum total T₄ and serum creatinine concentrations (with the total T₄ concentration above the normal range and the creatinine concentration within the normal range). Feeding food B for twelve weeks resulted in a decrease in both serum total T₄ and serum creatinine concentrations (with both total T₄ and creatinine concentrations falling within the normal ranges).

Unless otherwise staled, all percentages provided herein are weight percentages on a dry matter basis, e.g., 31% protein means 31% protein on a dry matter basis. Also, the concentrations of ingredients are given, e.g., 1 mg/kg iodine means 1 mg/kg iodine on a dry matter basis. The term “dry matter basis” means that an ingredient's concentration in a composition is measured after removing the moisture from the composition.

The singular forms “a”, “an”, and “the” include plural reference unless the context clearly indicates otherwise. The terms “comprise”, “comprises”, and “comprising” are to be interpreted inclusively rather than exclusively.

In the specification, there have been disclosed typical preferred embodiments of the invention and, although specific terms are employed, they are used in a generic and descriptive sense only and not for purposes of limitation, the scope of the invention being set forth in the claims. Obviously many modifications and variations of the invention are possible in light of die above teachings. It is therefore to be understood that within the scope of the appended claims the invention may be practiced otherwise than as specifically described.

Unless defined otherwise, all technical and scientific terms and acronyms used herein have the same meanings as commonly understood by one of ordinary skill in the art in the field of the invention. Although any compositions, methods, articles of manufacture, and means for communicating information similar or equivalent to the ones described herein can be used to practice die invention, the preferred compositions, methods, articles of manufacture, and means for communicating information are described herein.

All patents, patent applications, and publications cited herein are incorporated by reference into this patent application to the extent allowed by law. The discussion of those references is intended merely to summarize the assertions made by their authors. No admission is made that any such patents, patent applications, or publications (or a portion thereof) is relevant prior art. Applicants reserve the right to challenge the accuracy and pertinence of the cited patents, patent applications, and publications.

1. A method for preventing chronic renal failure in a feline with hyperthyroidism comprising feeding the feline a composition comprising:
from about 28 to about 35% protein on a dry matter basis wherein the protein comprises at least about 75% vegetable protein: and
from about 0.1 to less than about 1 mg/kg iodine on a dry matter basis and/or from about 0.1 to about 1 mg/kg selenium on a dry matter basis.

2. The method of claim 1 wherein the composition comprises from about 31 to about 35% protein.

3. The method of claim 1 or 2 wherein the composition comprises from about 0.1 to about 0.3 mg/kg iodine.

4. The method of claim 1 or 2 wherein the composition comprises from about 0.15 to about 0.65 mg/kg selenium.

5. The method of claim 1 or 2 wherein the composition comprises 0.1 to about 0.5 mg/kg iodine and from about 0.1 to about 0.8 mg/kg selenium.

6. The method of claim 1 wherein the composition further comprises an antithyroid agent.

7. The method of claim 1 wherein the composition further comprises from about 0.6 to about 0.8% phosphorus.

8. The method of claim 1 wherein the composition further comprises from about 0.25 to about 0.35% sodium.

9. The method of claim 1 wherein the composition further comprises from about 0.75 to about 1% potassium.

10. The method of claim 1 wherein the method further comprises administering to the feline an antithyroid agent in conjunction with feeding the feline the composition.

11. The method of claim 10 wherein the antithyroid agent comprises a thioureylene selected from the group consisting of methimazole, propylthiouracil, and carbimazole.

12. (canceled)

13. (canceled)

14. The method of claim 10 or claim 11 wherein a therapeutically-effective amount of the antithyroid agent is administered to the feline.

15. The method of claim 1 wherein the feline is a cat.

16. The method of claim 1 wherein the composition is a food composition.

17. A method for treating chronic renal failure in a feline with hyperthyroidism comprising feeding the feline a composition comprising:
from about 28 to about 30% protein on a dry matter basis wherein the protein comprises at least about 75% vegetable protein; and
from about 0.1 to less than about 1 mg/kg iodine on a dry matter basis and/or from about 0.1 to about 1 mg/kg selenium on a dry matter basis.

18. The method of claim 17 wherein the composition comprises from about 0.1 to about 0.3 mg/kg iodine.

19. The method of claim 17 or 18 wherein the composition comprises from about 0.15 to about 0.65 mg/kg selenium.

20. The method of claim 17 wherein the composition comprises 0.1 to about 0.5 mg/kg iodine and from about 0.1 to about 0.8 mg/kg selenium.

21. The method of claim 17 wherein the composition further comprises an antithyroid agent.

22. The method of claim 17 wherein the composition further comprises from about 0.45 to about 0.6% phosphorus.

23. The method of claim 17 wherein the composition further comprises from about 0.2 to about 0.25% sodium.

24. The method of claim 17 wherein the composition further comprises from about 0.75 to about 1% potassium.

25. The method of claim 17 wherein the method further comprises administering to the feline an antithyroid agent in conjunction with feeding the feline the composition.

26. The method of claim 25 wherein the antithyroid agent comprises a thioureylene selected from the group consisting of methimazole, propylthiouracil, and carbimazole.

27. (canceled)

28. (canceled)

29. The method of claim 25 or 26 wherein a therapeutically-effective amount of the antithyroid agent is administered to the feline.

30. The method of claim 17 wherein the method further comprises administering to the feline an anti-chronic renal failure agent.

31. The method of claim 30 wherein a therapeutically-effective amount of the anti-chronic renal failure agent is administered to the feline.

32. The method of claim 17 wherein the feline is a cat.

33. The method of claim 17 wherein the composition is a food composition.

34. A composition for preventing chronic renal failure in a feline with hyperthyroidism comprising:
from about 28 to about 35% protein on a dry matter basis wherein the protein comprises at least about 75% vegetable protein; and
from about 0.1 to less than about 1 mg/kg iodine on a dry matter basis and/or from about 0.1 to about 1 mg/kg selenium on a dry matter basis.

35. The composition of claim 34 wherein the composition comprises from about 31 to about 35% protein.

36. The composition of claim 34 or 35 wherein the composition comprises from about 0.1 to about 0.3 mg/kg iodine.

37. The composition of claim 34 or claim 35 wherein the composition comprises from about 0.15 to about 0.65 mg/kg selenium.

38. The composition of claim 34 or 35 wherein the composition comprises 0.1 to about 0.5 mg/kg iodine and from about 0.1 to about 0.8 mg/kg selenium.

39. The composition of claim 34 wherein the composition further comprises from about 0.6 to about 0.8% phosphorus.

40. The composition of claim 34 wherein the composition further comprises from about 0.25 to about 0.35% sodium.

41. The composition of claim 34 wherein the composition further comprises from about 0.75 to about 1% potassium.

42. The composition of claim 34 wherein the feline is a cat.

43. The composition of claim 34 wherein the composition is a food composition.

44. A composition for treating chronic renal failure in a feline with hyperthyroidism comprising:
from about 28 to about 30% protein on a dry matter basis wherein the protein comprises at least about 75% vegetable protein: and
from about 0.1 to less than about 1 mg/kg iodine on a dry matter basis and/or from about 0.1 to about 1 mg/kg selenium on a dry matter basis.

45. The composition of claim 44 wherein the composition comprises from about 0.1 to about 0.3 mg/kg iodine.

46. The composition of claim 44 or 45 wherein the composition comprises from about 0.15 to about 0.65 mg/kg selenium.

47. The composition of claim 44 wherein the composition comprises 0.1 to about 0.5 mg/kg iodine and from about 0.1 to about 0.8 mg/kg selenium.

48. The composition of claim 44 wherein the composition further comprises from about 0.45 to about 0.6% phosphorus.

49. The composition of claim 44 wherein the composition further comprises from about 0.2 to about 0.25% sodium.

50. The composition of claim 44 wherein the composition further comprises from about 0.75 to about 1% potassium.

51. The composition of claim 44 wherein the feline is a cat.

52. The composition of claim 44 wherein the composition is a food composition.

53. (canceled)

54. (canceled)

55. (canceled)

56. (canceled)

57. (canceled)

58. Use of vegetable protein in a food composition or in the manufacture of a food composition to treat and/or prevent chronic renal failure in a feline having hyperthyroidism, wherein the amount of protein in composition is from about 25% to about 35%) protein on a dry matter basis, which comprises at least about 75% vegetable protein.

59. Use of a combination of vegetable protein and iodine in the manufacture of a food composition to treat and/or prevent chronic renal failure in a feline having hyperthyroidism, wherein amount of protein in the composition is from about 25% to about 35% protein on a dry matter basis, the amount of iodine in the composition is from about 0.1 mg/kg to about 1 mg/kg iodine, and wherein the protein comprises at least about 75% vegetable protein.
